(19) United States
(12) Patent Application Publication
(10) Pub. No.: US 2011/0224232 A1
(43) Pub. Date: Sep. 15, 2011

(54) Treatment of Pulmonary Fungal Infection With Voriconazole via Inhalation

(75) Inventors: Robert O. Williams III, Austin, TX (US); Rupert O. Zimmerer, Lawrence, KS (US); Jason T. MxConville, Austin, TX (US); Justin A. Tolman, Papillion, NE (US); Nathan P. Wiederhold, San Antonio, TX (US); Jay I. Peters, San Antonio, TX (US)

(73) Assignees: Board of Regents, The University of Texas System, Austin, TX (US); CyDex Pharmaceuticals, Inc., Lenexa, KS (US)

(21) Appl. No.: 12/991,403

(22) PCT Filed: May 6, 2009
(86) PCT No.: PCT/US2009/043027

§ 371 Date: May 11, 2011

Related U.S. Application Data
(60) Provisional application No. 61/050,918, filed on May 6, 2008.

(51) Int. Cl. A61K 31/506; A61P 31/10

(52) U.S. Cl. 514/256

(57) ABSTRACT

A method of treating fungal infection by pulmonary administration of a solution of voriconazole and cyclodextrin is provided The fungal infection can be a pulmonary infection. The solution can be an inhalable aqueous formulation that can be administered via the mouth or nose. The cyclodextrin can be a water soluble cyclodextrin derivative such as sulfoalkyl ether cyclodextrin. The formulation can be administered via a spray device or nebulizer.

### TECHNICAL FIELD OF THE INVENTION

The present invention concerns a method of treating fungal infection by administration of voriconazole via inhalation. More particularly, the invention concerns the administration of an aqueous inhalable formulation of voriconazole and cyclodextrin for the treatment of aspergillosis.

### BACKGROUND ART

Systemic fungal infections (SFI) are becoming more prevalent in the United States healthcare system due to a higher incidence of immuno-compromised patients following improvements in transplantation, cancer chemotherapy, and antiretroviral therapy. These serious fungal infections can lead to longer hospital stays, increased health care costs, and ultimately high patient mortality. Although infections due to Candidia species (spp.) are more common, the mortality associated with invasive Aspergillus spp. (Invasive Aspergillosis, Iowa) is much higher despite treatment.

Aspergilli are a group of fungi ubiquitous in nature and easily cultured from air, water, soil, vegetation, and any site where dust accumulates. In appropriate conditions the organism forms large amounts of spores which are released into the environment where they may remain suspended for long periods. Aspergillus spores are small (2.5 to 3.5 microns in diameter) and easily inhaled where they may colonize the upper or lower airways. IA is primarily caused by the inhalation and subsequent germination of conidia by patients with suppressed immune responses. Therefore, the primary site of infection is the lungs, although dissemination to other organs and other sites of infection can occur. Several hundred species of Aspergillus exist with three causing the majority of disease in humans, A fumigatus and A. flavus and A. terreus.

Several clinical manifestations of Aspergillus spp. pulmonary infection occur. These include an allergic syndrome (allergic bronchopulmonary aspergillosis), fungus ball formation in preexisting lung cavities and invasive pulmonary aspergillosis. Aspergillus pneumonia results from fungal invasion of hyphae into the lung tissue. From the lung the fungus may disseminate through the blood stream to the brain, kidney, liver, heart and other sites.

In a study conducted by NationMaster.com, the mortality statistics for number of deaths caused by invasive asergillosis varies country to country from about 0.02 to 3.3 per million people. Its treatment is difficult and once infected patient prognosis is poor. It is especially harmful in immunocompromised patients, lung transplant patients, chemotherapy patients, and elderly patients.

In highly immunocompromized hosts Aspergillus spp. causes severe opportunistic infections that carry a high mortality. Although invasive aspergillosis may be community acquired, most cases are nosocomial in origin. Major outbreaks of invasive nosocomial aspergillosis have been reported associated with hospital construction, renovation and maintenance, activities that allow spores to become airborne.

Treatment options for IA include amphotericin B and the triazole antifungal agents. Although these agents have excellent in vitro activity, their in vivo activity is limited in many instances by their poor bioavailability due to poor aqueous solubility and/or dose-limiting toxicities. In 2002, a controlled clinical trial established voriconazole (VFEND® IV) as the first line therapy for IA. Voriconazole IV is available as an inclusion complex of the active pharmaceutical ingredient (API) with CAPTISOL® (sulfobutyl ether-β-cyclodextrin). The cyclodextrin functions primarily as an aqueous solubilizer. The formulation is administered parenterally as a clear aqueous liquid comprising SAE-CD and voriconazole for the treatment of pulmonary fungal infection.

Other antifungal agents have shown reductions in fungal burden using the model of invasive pulmonary aspergillosis disclosed below. A recent study demonstrated significant reductions in fungal burden as measured by quantitative real-time PCR with high doses of posaconazole (40 mg/kg per day) (Wiederhold et al. Antimicrob. Agents Chemother. 2008 52: 1176).

In vitro studies have shown a dose response relationship of increased activity of voriconazole against A. fumigatus with increasing drug concentrations. One study reported effective inhibition of growth with an EC50 value (concentration resulting in 50% inhibition of growth compared to control) of 0.18 micrograms/mL as determined by non-linear regression analysis, and fungicidal activity at a low concentration (0.5 microgram/mL) (Lewis et al. Antimicrob Agents Chemother 2005; 49: 945). Clinically, significant interpatient and intrapatient pharmacokinetic variability has been reported in several studies, irrespective of the mg/kg dose (Pascual et al. Clin Infect Dis 2008: 46: 201, Trifilio et al. Bone Marrow Trans 2005; 35: 509, Trifilio et al. Cancer 2007; 109: 1532). What has been observed in a clinical study is that patients with voriconazole trough concentrations≦1 microgram/mL are more likely to experience clinical failure (Pascual et al. Clin Infect Dis 2008: 46: 201). When the dose of voriconazole was increased in these patients and trough levels were increased to >1 microgram/mL, each of these patients responded. Similarly, a second study reported reduced response rates in patients with random voriconazole plasma concentrations of <2.05 microgram/mL (Smith et al. Antimicrob Agents Chemother 2006; 50: 1570).

Previous animal models of invasive fungal infections have demonstrated AUC to MIC ratio to be the PK/PD parameter that is predictive of efficacy (Andes et al. in Antimicrob. Agents Chemother. (2003) 47:3165).

In humans, recent data have indicated that efficacy of voriconazole is associated with plasma trough concentrations of >1 microgram/mL (Pascual et al. in Clin. Infect. Dis. (2008), 46:201), or random plasma concentrations>2.05 microgram/mL (Smith et al. in Antimicrob. Agents Chemother. (2006) 50:1570).

U.S. Pat. No. 6,632,803 and PCT International Publication No. WO 98/58677 to Harding discloses clear aqueous liquid formulations comprising voriconazole and a SAE-CD. They are indicated for parenteral, in particular i.v., administration to a subject.

U.S. Publication No. 20050186267 to CyDex, Inc. discloses capsule formulations containing an aqueous fill comprising SAE-CD and a drug.

PCT International Publication No. WO 2006/026502 discloses an inhalable formulation containing respirable aggregates of voriconazole, among other suitable drugs. The publication discourages the use of cyclodextrins due to potential hepatotoxicity.

Various publications disclose the pulmonary administration of antifungal agents for treating pulmonary fungal infection, for example, PCT International Publication No. WO 2006/108556 and No. 2004/060903, U.S. Publications No. 20050244339, No. 20070196461 to Weers, No. 20070202051 to Schuschnig, No. 20070178166 to Bernstein, No. 20060257491 to Morton, No. 20050244339 to Jauernig, No. 20070082870 to Buchanan et al., and No. 20040176391 to Weers, European Publications No. EP0982031 to Pfizer and No. EP 1820493 to Pari Pharma GMBH.

A need remains for an effective therapy for invasive pulmonary fungal infections to improve the current poor prognosis of patients suffering from this deadly disease. None of the known art discloses the claimed method of treating pulmonary fungal infection with an aqueous solution formulation of cyclodextrin and voriconazole.

### DISCLOSURE OF THE INVENTION

The present invention provides an aqueous liquid formulation comprising voriconazole, SAE-CD and an aqueous liquid carrier for use in the treatment of fungal infection. Some embodiments of the invention require a clear liquid formulation; although, a suspension formulation might also be used. The method requires pulmonary administration of the formulation, which can be either by the mouth or the nose of a subject, via a nebulizer or other type of aerosol-generating device. The inhalable liquid formulation comprises a therapeutically effective amount of voriconazole, aqueous liquid carrier, and cyclodextrin derivative.

The present invention also provides a method of treating, preventing or reducing the occurrence of a disease, disorder or condition having an etiology associated with fungal infection or of a disease, disorder or condition that is therapeutically responsive to voriconazole therapy, the method comprising administering the formulation of the invention to a subject in need thereof via pulmonary administration.

The invention also provides a method of treating a disease, condition or disorder comprising administering to a subject in need thereof: a therapeutically effective amount of voriconazole in a composition or formulation of the invention, and a therapeutically effective amount of a second therapeutic agent, such as described herein. The second therapeutic agent may or may not be included in the same composition or formulation as the voriconazole.

The invention also provides a method of treating, preventing or reducing the occurrence of a disease, disorder or condition having an etiology associated with fungal infection or of a disease, disorder or condition that is therapeutically responsive to voriconazole therapy, the method comprising: administering to a subject in need thereof via pulmonary administration a therapeutically effective amount of voriconazole in an inhalable aqueous liquid formulation comprising voriconazole, sulfoalkyl ether cyclodextrin and an aqueous liquid carrier.

In some embodiments, the invention provides a method of treating a fungal infection in a subject comprising administering via inhalation to a subject in need thereof a therapeutically effective dose of an inhalable aqueous liquid formulation comprising sulfoalkyl ether cyclodextrin, voriconazole, and aqueous liquid carrier, wherein the dose comprises 0.5 to 10 ml, 0.5 to 1 ml, 1 to 3 ml, >3 to 6 ml, >6 to 10 ml, 0.25 to 20 ml, 0.1 to 50 ml, or 0.1 to 100 ml of formulation containing 1 to 10 mg/ml, 1 to 2.5 mg/ml, >2.5 to 5 mg/ml, >5 to 7.5 mg/ml, >7.5 to 10 mg/ml, 1 to 15 mg/ml, 0.75 to 20 mg/ml, 0.5 to 25 mg/ml, 0.25 to 30 mg/ml, or 0.1 to 50 mg/ml of voriconazole completely or partially nebulized over 1 to up to 120 minutes. It can be readily recognized by those of skill in the art that the nebulization time could be varied up to, for example, 12 hours, depending on such factors as condition of the patient, severity of the infection, and the like. This dose could provide in the plasma of the subject a Cmax in the range of about 2 to 8 μg/mL, 2 to 4 μg/mL, >4 to 6 μg/mL, >6 to 8 μg/mL, 1.5 to 10 μg/mL, 1.25 to 15 μg/mL, or 1 to 20 μg/mL, and an AUC in the range of about 1 to 100 μg·hr/mL, 0.5 to 200 μg·hr/mL, 1 to 50 μg·hr/mL, or >50 to 100 μg·hr/mL. In some embodiments, the method is limited to treatment of pulmonary fungal infection. The formulation can be administered such that it provides a Tmax in the lung in the range of about 1-60 min and a Tmax in the blood in the range of about 5-120 min. In some embodiments, the inhalable formulation is administered over a 1 up to 120 minute period once to four times daily. The total daily dose can be divided among one to four unit doses, meaning a unit dose of the formulation can be administered once to four times daily. The formulation can be administered such that it provides a total daily dose of about 0.01 to 6 mg of voriconazole per kg of body weight. In some embodiments, a single dose of inhaled voriconazole could consist of 2.5 to 4 mL of formulation containing 6.25 mg/mL of voriconazole. In some embodiments, a unit dose of the formulation is administered over a period of about 15 min or no more than 20 min or no less than 5 min. In some embodiments, an acute dose of the formulation is administered and in other embodiments the dose is administered chronically.

The invention also provides a method of treating a fungal infection in a subject comprising: administering via inhalation to a subject in need thereof a therapeutically effective amount of voriconazole in an inhalable aqueous liquid formulation comprising sulfoalkyl ether cyclodextrin, voriconazole, and aqueous liquid carrier, wherein the dose comprises 0.5 to 10 ml, 0.5 to 1 ml, 1 to 3 ml, >3 to 6 ml, >6 to 10 ml, 0.25 to 20 ml, 0.1 to 50 ml, or 0.1 to 100 ml of formulation; and the formulation comprises voriconazole present at a concentration of 1 to 10 mg/ml, 1 to 2.5 mg/ml, >2.5 to 5 mg/ml, >5 to 7.5 mg/ml, >7.5 to 10 mg/ml, 1 to 15 mg/ml, 0.75 to 20 mg/ml, 0.5 to 25 mg/ml, 0.25 to 30 mg/ml, or 0.1 to 50 mg/ml of formulation.

The cyclodextrin derivative is present in an amount sufficient to dissolve the voriconazole such that at least 50% wt., at least 75% wt., at least 90% wt., at least 95% wt., at least 97.5% wt., or substantially all of the voriconazole is dissolved. The formulation can be a clear or substantially clear solution containing less than about 20% wt. of solids. In some embodiments, the cyclodextrin derivative is a sulfoalkyl ether cyclodextrin (SAE-CD) compound or mixture of sulfoalkyl ether cyclodextrin compounds. In some embodiments, the pH of the formulation is in the range of 4 to 9, 5 to 8, or 5.5 to 7.5. The molar ratio of SAE-CD to voriconazole can be at least 0.5:1, at least 0.7:1, at least 0.9:1, at least 1:1, at least 1.2:1, at least 1.5:1, at least 1.75:1, at least 1.9:1, at least 2:1, at least 2.1:1, at least 2.2:1, at least 2.4:1, at least 2.5:1, at least 2.75:1, at least 3:1, or at least 4:1; range from 2:1 to 10:1, from 0.5:1 to 20:1, 0.7:1 to 15:1, 1:1 to 12:1, 1.5:1 to 10:1; and/or be less than 20:1, less than 15:1, less than 12:1, less than 11:1, less than 10:1, less than 9:1, less than 8:1, less than 7:1, less than 6:1, or less than 5:1. In some embodiments, the formulation is a modified version of the VFEND® formulation. The VFEND® IV formulation can be reconstituted as instructed in the product literature with an appropriate diluent, including sterile water for injection (SWFI), such that the voriconazole concentration is 10 mg/mL. The reconstituted VFEND® IV formulation can then be diluted with an appropriate diluent, including SWFI, to a concentration less than 10 mg/ml. In some embodiments, the voriconazole concentration can be 6.25 mg/mL.

The inhalable formulation can be administered via the mouth or nose ultimately for pulmonary delivery thereof. Devices suitable for such pulmonary delivery include nebulizers, dry powder inhalers, and metered-dose inhalers. In some embodiments, the inhalable formulation can be administered by air-jet, ultrasonic, or vibrating-mesh nebulizers and can include Pari LC Star, Aeroeclipse II, Prodose (HaloLite), Acorn II, T Up-draft II, Sidestream, AeroTech II, Mini heart, MisterNeb, Sonix 2000, MABISMist II and other suitable aerosol systems. In some embodiments, the nebulizer is a vibrating-mesh nebulizer that could include an AERONEB PRO, AERONEB SOLO, AERONEB GO, AERONEB LAB, OMRON MICROAIR, PARI EFLOW, RESPIRONICS I-NEB, or other suitable devices.

The method of the invention is such that it provides an improved clinical effect as compared to the pulmonary administration of an otherwise similar control sample comprising itraconazole instead of voriconazole. In some embodiments, the method and formulation of the invention together provide an improved method for the treatment of pulmonary fungal infection in a mammal.

The invention includes all combinations of aspects, embodiments and sub-embodiments of the invention disclosed herein.

### DESCRIPTION OF THE DRAWINGS

The following figures form part of the present description and describe exemplary embodiments of the claimed invention. The skilled artisan will, in light of these figures and the description herein, be able to practice the invention without undue experimentation.

FIG. 1A depicts a top plan view of the nose-only dosing chamber used to evaluate the method and formulation of the invention on mice.

FIG. 1B depicts a side elevation view of the nose-only dosing chamber of FIG. 1A.

FIG. 2 depicts the timeline for a prophylaxis study conducted to establish therapeutic efficacy of the method and formulation according to the invention. VOR—Voriconazole (Inhalation—BID 30-40 mg/kg), Control—Captisol (Inhalation—BID), AmB—Amphotericin B deoxycholate (Intraperitoneal—QD 1 mg/kg).

FIG. 3 depicts a plot of the concentration of VFEND® IV solutions (N=10) versus osmolality as determined using a μOsmette micro-osmometer (Precision Systems Inc., Natick, Mass.). Error bars represent one standard deviation.

FIG. 4 depicts a plot of time versus concentration of inhaled voriconazole formulation in the lungs of male ICR mice following 20 minute nebulization, which is equivalent to an approximate dose of about 30-40 mg of voriconazole/kg of body weight. The number of mice was 2-4 (** denotes N=4) per time point. Lungs were harvested and homogenized to determine drug concentration. Error bars represent one standard deviation. The data was obtained following administration of a single dose.

FIG. 5 depicts plot of time versus concentration of inhaled voriconazole formulation in the plasma of male ICR mice following 20 minute nebulization, which is equivalent to an approximate dose of about 30-40 mg of voriconazole/kg of body weight. The number of mice was 1-4 (* denotes N=1, ** denotes N=4) per time point. Plasma was separated from whole blood collected to determine drug concentration. Error bars represent one standard deviation. The data was obtained following administration of a single dose.

FIG. 6 depicts a plot of survival days after inoculation with Aspergillus fumigatus versus the percentage of infected female ICR mice (N=12 for each of three groups: VOR=Voriconazole (Inhalation—BID 30-40 mg/kg), Control=Captisol (Inhalation—BID), AmB=Amphotericin B deoxycholate (Intraperitoneal—QD 1 mg/kg)) surviving following 20 minute nebulization using a 1 L/min flow rate. Treatment continued from Day 0 to Day +7. Survival of A. fumigatus—infected female ICR mice, 20 minute nebulization, 1 L/min flow rate. Immunosuppression occurred on Day −2 and Day +3. Inoculation occurred on Day 0. N=12 for each of three groups: Voriconazole Inhalation administered BID, Control was administered CAPTISOL® Inhalation BID, Amphotericin B deoxycholate was administered by intraperitoneal injection QD at 1 mg/kg. Treatment continued from Day −2 through Day 7 for voriconazole and control groups and Day 0 through Day +7 for the amphotericin B group.

FIGS. 7A-7D depict charts of the pulmonary fungal burden of mice following inoculation with A. fumigatus (FIG. 7A—Day +8 fungal burden as determined by colony forming units (CFU) compared to the fungal burden at 1 hour post infection; FIG. 7B—Summary fungal burden by CFU for all samples taken, day 8 and day 12 compared with 1 hour post infection; FIG. 7C—Day +12 fungal burden by real-time quantitative PCR as measured in conidial equivalents; FIG. 7D—Day +12 fungal burden by real-time quantitative PCR as measured in conidial equivalents normalized for wet lung mass.)

FIGS. 8A-8B depicts visual microscopy images of mouse lung tissue after infection with A. fumigatus conidia and treatment with voriconazole (FIG. 8A) and amphotericin B (FIG. 8B).

FIG. 9 depicts a phase solubility diagram for voriconazole in the presence of varying amounts of sulfoalkyl ether cyclodextrin.

FIG. 10 depicts a plot of plasma concentration of voriconazole verse time for a multidose pharmacokinetic profile after administration of voriconazole via inhalation to mice. Male ICR mice, 20 minute nebulization. N=6 mice per time point. Trough samples obtained immediately before the next scheduled dose on days 3, 5, 10, and 12. Peak samples obtained 30 minutes after the completion of nebulization on day 5. Plasma separated from whole blood collected to determine drug concentration. Error bars represent one standard deviation. Red line indicates the MIC₉₀ (0.52 μg/mL). Blue line indicates the MIC₅₀ (0.25 μg/mL)

FIGS. 11A-11D depict images of obtained from lung sections of mice infected with A. fumigatus. Lungs were harvested from two animals per treatment group on Day +8 and Day +12 following inoculation. Photomicrographs of lung sections, focusing on pulmonary lesions and/or abnormal histological findings, were taken for each animal sampled at a magnification of 10×. The photomicrographs are arranged by treatment group and the day of sampling. Images are also further identified by a sequential number that was assigned when each animal was randomly selected for sacrifice.

FIGS. 12A-12B depicts charts for the distribution of pulmonary lesions in histological samples from tissue obtained from treated and untreated mice.

### DESCRIPTION OF THE INVENTION

Any form of voriconazole (2-(2,4-difluorophenyl)-3-(5-fluoropyrimidin-4-yl)-1-(1H-1,2,4-triazol-1-yl)butan-2-ol) can be used according to the invention. Voriconazole can also be made according to U.S. Pat. No. 5,116,844, No. 5,364,938, No. 5,567,817, No. 5,773,443 and other such methods. Voriconazole is commercially available from Pfizer, Inc. (New York, N.Y., USA).

The formulation of the invention is inhalable, meaning it can be administered to the respiratory tract. The formulation is an aqueous formulation comprising water soluble cyclodextrin derivative, an aqueous carrier, and voriconazole. The formulation can be made by modification of a sample of commercially available VFEND® formulation. VFEND® IV is reconstituted with SWFI to a voriconazole concentration of 10 mg/mL. The reconstituted formulation is then diluted with SWFI to a voriconazole concentration of 6.25 mg/mL. Voriconazole is available as an aqueous liquid formulation comprising CAPTISOL SAE-CD and voriconazole under the trademark VFEND® (Pfizer, Inc.). The formulation can be made as described in U.S. Pat. No. 6,632,803. Alternatively, the formulation can be made as described herein and/or as follows: Method 1—to an aqueous liquid carrier is added the water soluble cyclodextrin derivative and voriconazole; Method 2—to an aqueous liquid carrier is added the water soluble cyclodextrin derivative and then voriconazole; Method 3—to an aqueous liquid carrier comprising water soluble cyclodextrin derivative is added voriconazole; Method 4—to an aqueous liquid carrier comprising a suspension of voriconazole is added the water soluble cyclodextrin derivative.

Derivatized cyclodextrins suitable in the invention include water soluble derivatized cyclodextrins. The water soluble cyclodextrin derivative compositions used to make the combination composition of the invention can be comprise sulfoalkyl ether cyclodextrin (SAE-CD) derivatives (such as CAPTISOL® and ADVASEP®) available from CyDex, Inc. (Lenexa, Kans., USA). It is available in a variety of grades varying in physical morphology, degree of substitution, salt form, parent cyclodextrin content, and cyclodextrin ring size.

A water soluble cyclodextrin derivative composition can comprise a SAE-CD compound, or mixture of compounds, of the Formula 1:

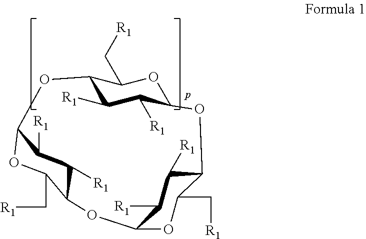

Wherein p is 4, 5 or 6; R₁ is independently selected at each occurrence from —OH or -SAET; -SAE is a —O—(C₂-C₆ alkylene)-SO₃⁻ group, wherein at least one SAE is independently a —O—(C₂-C₆ alkylene)-SO₃⁻ group, preferably a —O—(CH₂)_{g}SO₃⁻ group, wherein g is 2 to 6, preferably 2 to 4, (e.g. —OCH₂CH₂CH₂SO₃⁻ or —OCH₂CH₂CH₂CH₂SO₃⁻); and T is independently selected at each occurrence from the group consisting of pharmaceutically acceptable cations, which group includes, for example, H⁺, alkali metals (e.g. Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine, ethylenediamine and (C₄-C₈)-cycloalkanolamine among others; provided that at least one R₁ is -SAET.

The terms “alkylene” and “alkyl,” as used herein (e.g., in the —O—(C₂-C₆-alkylene)SO₃⁻ group or in the alkylamines cations), include linear, cyclic, and branched, saturated and unsaturated (i.e., containing one double bond) divalent alkylene groups and monovalent alkyl groups, respectively. The term “alkanol” in this text likewise includes both linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl)cyclic alcohols.

The cyclodextrin derivatives can differ in their degree of substitution by functional groups, the number of carbons in the functional groups, their molecular weight, the number of glucopyranose units contained in the base cyclodextrin used to form the derivatized cyclodextrin and or their substitution patterns. In addition, the derivatization of a cyclodextrin with functional groups occurs in a controlled, although not exact manner. For this reason, the degree of substitution is actually a number representing the average number of functional groups per cyclodextrin (for example, SBE7-β-CD, has an average of 7 substitutions per cyclodextrin). Thus, it has an average degree of substitution (ADS) of about 7. In addition, the regiochemistry of substitution of the hydroxyl groups of the cyclodextrin is variable with regard to the substitution of specific hydroxyl groups of the hexose ring. For this reason, substitution of the different hydroxyl groups is likely to occur during manufacture of the derivatized cyclodextrin, and a particular derivatized cyclodextrin will possess a preferential, although not exclusive or specific, substitution pattern. Given the above, the molecular weight of a particular derivatized cyclodextrin composition may vary from batch to batch.

A cyclodextrin derivative composition comprises a distribution of plural individual species, each species having an individual degree of substitution (IDS). The content of each of the cyclodextrin species in a particular composition can be quantified using capillary electrophoresis. CAPTISOL® is a water soluble cyclodextrin derivative comprising a distribution of individual sulfobutyl ether cyclodextrin derivative species.

In a single parent CD molecule, there are 3v+6 hydroxyl moieties available for derivatization. Where v=4 (α-CD), “y” the degree of substitution for the moiety can range in value from 1 to 17. Where v=5 (β-CD), “y” the degree of substitution for the moiety can range in value from 1 to 20. Where v=6 (γ-CD), “y” the degree of substitution for the moiety can range in value from 1 to 23. In general, “y” also ranges in value from 1 to 3v+g, where g ranges in value from 0 to 5. “y” may also range from 1 to 2v+g, or from 1 to 1v+g.

The degree of substitution (DS) for a specific moiety (SAE, for example) is a measure of the number of SAE substituents attached to an individual CD molecule, in other words, the moles of substituent per mole of CD. Therefore, each substituent has its own DS for an individual CD derivative species. The average degree of substitution (ADS) for a substituent is a measure of the total number of substituents present per CD molecule for the distribution of CD derivatives within a CD derivative composition of the invention. Therefore, SAE4.0-CD has an ADS (per CD molecule) of 4.0.

Within a given CD derivative composition or combination composition, the substituents of the CD derivative(s) thereof can be the same. For example, SAE moieties can have the same type of alkylene (alkyl) radical upon each occurrence in a CD derivative composition. In such an embodiment, the alkylene radical in the SAE moiety might be ethyl, propyl, butyl, pentyl or hexyl in each occurrence in a CD derivative composition.

When at least one R₁ in the CD molecule is -SAET, the degree of substitution, in terms of the -SAET moiety, is understood to be at least one. The term SAE is used to denote a sulfoalkyl (alkylsulfonic acid) ether moiety it being understood that the SAE moiety comprises a cation (T) unless otherwise specified. Accordingly, the terms SAE and SAET may, as appropriate, be used interchangeably herein.

Further exemplary SAE-CD derivatives include:

| SAEx-α-CD | SAEx-β-CD | SAEx-γ-CD |
|---|---|---|
| SEEx-α-CD | SEEx-β-CD | SEEx-γ-CD |
| SPEx-α-CD | SPEx-β-CD | SPEx-γ-CD |
| SBEx-α-CD | SBEx-β-CD | SBEx-γ-CD |
| SPtEx-α-CD | SPtEx-β-CD | SPtEx-γ-CD |
| SHEx-α-CD | SHEx-β-CD | SHEx-γ-CD |

wherein SEE denotes sulfoethyl ether, SPE denotes sulfopropyl ether, SBE denotes sulfobutyl ether, SPtE denotes sulfopentyl ether, SHE denotes sulfohexyl ether, and x denotes the average degree of substitution. The salts thereof (with “T” as cation) are understood to be present.

Since SAE-CD is a poly-anionic cyclodextrin, it can be provided in different salt forms. Suitable counterions include cationic organic atoms or molecules and cationic inorganic atoms or molecules. The SAE-CD can include a single type of counterion or a mixture of different counterions. The properties of the SAE-CD can be modified by changing the identity of the counterion present. For example, a first salt form of SAE-CD can have a greater water activity reducing power than a different second salt form of SAE-CD. Likewise, an SAE-CD having a first degree of substitution can have a greater water activity reducing power than a second SAE-CD having a different degree of substitution.

The SAE-CD derivative that can be used as a starting material for preparing the combination composition is described in U.S. Pat. No. 5,376,645 and No. 5,134,127 to Stella et al, the entire disclosures of which are hereby incorporated by reference. According to one embodiment, the SAE-CD is SBE7-β-CD (CAPTISOL® cyclodextrin), or SBE4-β-CD (ADAVASEP®). An SAE-CD made according to other known procedures should also be suitable for use in the invention. Parmerter et al. (U.S. Pat. No. 3,426,011), Lammers et al. (Recl. Trav. Chim. Pays-Bas (1972), 91(6), 733-742); Staerke (1971), 23(5), 167-171), Qu et al. (J. Inclusion Phenom. Macro. Chem., (2002), 43, 213-221), Yoshinaga (Japanese Patent No. JP 05001102; U.S. Pat. No. 5,241,059), Zhang et al. (PCT International Publication No. WO 01/40316), Adam et al. (J. Med. Chem. (2002), 45, 1806-1816), and Tarver et al. (Bioorganic & Medicinal Chemistry (2002), 10, 1819-1827) disclose other suitable sulfoalkyl ether derivatized cyclodextrins for use as starting materials in preparing a combination composition according to the invention. A suitable SAE-CD starting material can be made according to the disclosure of Stella et al., Parmerter et al., Lammers et al., Qu et al., Yoshinaga, Zhang et al., Adam et al. or Tarver et al. A suitable SAE-CD can also be made according to the procedure(s) described herein.

A water soluble CD derivative composition possesses greater water solubility than the corresponding parent cyclodextrin from which it is made. The underivatized parent cyclodextrins α-CD, β-CD or γ-CDs are commercially available from WACKER BIOCHEM CORP. (Adrian, Mich.) and other sources. The parent cyclodextrins have limited water solubility as compared to SAE-CD. Underivatized α-CD has a water solubility of about 14.5% w/w at saturation. Underivatized β-CD has a water solubility of about 1.85% w/w at saturation. Underivatized γ-CD has a water solubility of about 23.2% w/w at saturation.

The water soluble cyclodextrin derivative composition is optionally processed to remove the major portion of the underivatized parent cyclodextrin or other contaminants.

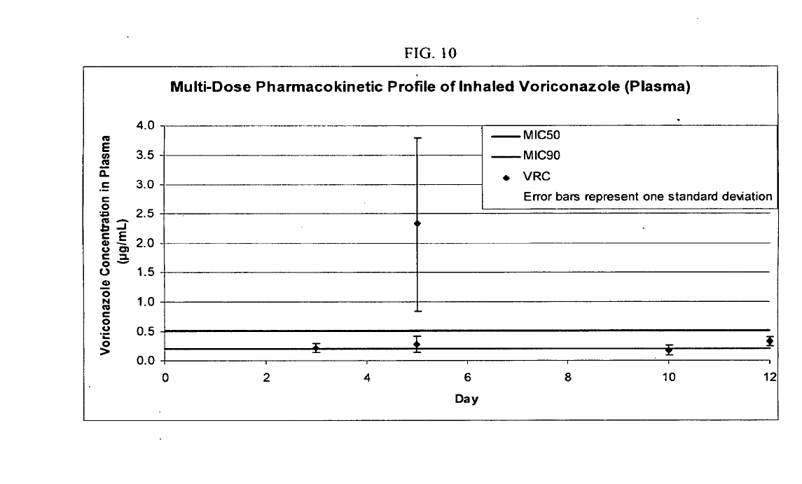

In the absence of a water soluble cyclodextrin derivative, voriconazole has an aqueous solubility of about 0.68-0.69 mg/ml in water at room temperature. The solubility of voriconazole in aqueous medium is increased by addition of water soluble cyclodextrin derivative in the formulation. FIG. 9 depicts a phase solubility diagram for voriconazole in water in the presence of varying amounts of SAE-CD. The area below the phase solubility curve denotes the region where the voriconazole is solubilized in an aqueous liquid medium to provide a substantially clear aqueous solution. In this region, the SAE-CD is present in molar excess of the voriconazole and in an amount sufficient to solubilize, and optionally stabilize, the voriconazole present in the liquid carrier. The boundary defined by the phase solubility curve will vary according to the amount or concentration of voriconazole and SAE-CD within a composition or formulation of the invention. The table below provides a summary of the minimum molar ratio of SAE-CD to voriconazole required to achieve the saturated solubility of the voriconazole in the composition or formulation of the invention under the conditions studied.

| | | Approximate Molar Ratio at Saturated |
|---|---|---|
| Voriconazole | SAE-CD | Solubility of Voriconazole |
| (mg/ml) | (mg/ml) | (SAE-CD:VOR) |
| 3.34 | 43.26 | 2.09 |
| 8.39 | 108.15 | 2.08 |
| 17.32 | 216.3 | 2.02 |
| 26.49 | 324.45 | 1.98 |
| 35.11 | 43.26 | 2.09 |

Based upon that data, the minimum molar ratio of SAE-CD to voriconazole required to provide a substantially clear solution is about 2:1. For embodiments of the invention where the formulation is a substantially clear solution, the molar ratio of SAE-CD to voriconazole will exceed 2:1 by at least 1%, at least 2%, at least 2.5%, at least 5%, at least, 7.5%, at least 10%, at least 12.5%, at least 15%, at least 17.5%, or at least 20%.

The molar ratio can be as described herein or less than 2:1 if a suspension formulation is desired. A suspension may provide a sustained release or extended pulmonary absorption period of the voriconazole. Higher ratios may be desirable from a manufacturing point of view and may result in a more robust formulation.

As used herein as regards a method of treatment of a subject, the term “treat”, “treatment” or “treating” means to alleviate, ameliorate, eliminate, reduce the severity of, reduce the frequency of, occurrence of, or prevent symptoms associated with a disease, disorder or condition having fungal infection as an etiological component. For the purposes of this invention, the term “treatment” is also intended to mean the use, administration, or application of the inhalable liquid formulation comprising a therapeutically effective amount of voriconazole, aqueous liquid carrier, and cyclodextrin derivative for an illness, injury, or disease or to prevent an illness, injury, or disease caused by or resulting from a fungal species.

As used herein, the term “therapeutically responsive to voriconazole” means that treatment of a subject with such a disease, disorder or condition with a therapeutically effective amount of voriconazole will result in a clinical benefit or therapeutic benefit in the subject. The method of treating, preventing, ameliorating, reducing the occurrence of, or reducing the risk of occurrence of a disease, disorder or condition that is therapeutically responsive to voriconazole therapy in a subject comprises administering to the subject in need thereof, via pulmonary administration, a formulation or composition of the invention, wherein the formulation or composition comprises SAE-CD and a dose of voriconazole. A therapeutically effective amount of voriconazole can include one, two, or more doses of voriconazole.

The term “unit dosage form” is used herein to mean a single dosage form containing a quantity of the active ingredient and the diluent or carrier, said quantity being such that one or more predetermined units are normally required for a single therapeutic administration. In the case of multi-dose forms, such as liquid-filled bottles, said predetermined unit will be one fraction such as a half or quarter of the multiple dose form. It will be understood that the specific dose level for any patient will depend upon a variety of factors including the indication being treated, therapeutic agent employed, the activity of therapeutic agent, severity of the indication, patient health, age, sex, weight, diet, and pharmacological response, the specific dosage form employed and other such factors.

The method of treatment of the invention can be used for treatment of any disease or disorder caused by a fungal genus, species or strain whose growth is inhibited by voriconazole. Exemplary diseases or disorders include infection with invasive Aspergillus spp., Candida spp., Fusarium spp., Pseudallescheria spp., Scedosporium spp., and yeast and yeast-like species, monilaceous moulds, dimorphic fungi, and dematiaceous fungi.

Species whose growth is inhibited by voriconazole include Aspergillus species (containing A. awamori, A. clavatus, A. flavus, A. fischeri, A. fumigatus, A. glaucus, A. heterothallicus, A. nidulans, A. niger, A. oryzae, A. repens, A. rubber, A. terreus, A. ustus, A. versicolor), Candida species (containing C. albicans, C. cifferii, C. dubliniensis, C. famata, C. glabrata, C. guilliermondii, C. kefyr, C. krusei, C. lambica, C. lipolytica, C. lusitaniae, C. parapsilosis, C. rugosa, C. stellatoidea, C. tropicalis), Fusarium species (containing F. moniliforme, F. oxysporum, F. proliferatum, F. solani), Pseudallescheria species (containing P. boydii, S. aurantiacum, P. minutispora, P. angusta, P. fusoidea, P. ellipsoidea, Cryptic species of clades 3 and 4), and Scedosporium species (containing S. apiospermum, S. prolificans). Other fungi whose growth is inhibited by voriconazole include yeast and yeast-like species (containing Blastoschizomyces capitatus, Cryptococcus neoformans, Cryptococcus gattii, Hansenula anomala, Rhodotorula rubra, Saccharomyces cerevisiae, Sporobolomyces salmonicolor, Trichosporon asahii, Trichosporon beigelii, Trichosporon capitatum, Trichosporon cutaneum, Trichosporon inkin, Trichosporon mucoides, Trichosporon ovoides), Monilaceous moulds (containing Acremonium alabamensis, Acremonium strictum, Scopulariopsis brumptii, Paecilomyces lilacinus, Trichoderma longibrachiatum), Dimorphic fungi (containing Blastomyces dermatitidis, Coccidioides immitis, Histoplasma capsulatum, Paracoccidioides brasiliensis, Penicillium marneffei, Sprothrix schenckii), and Dematiaceous fungi (containing Alternaria alternata, Alternaria pullulans, Alternaria tenuis, Aureobasidium pullulans, Bipolaris australiensis, Bipolaris hawaiiensis, bipolaris spicifera, Botryomyces caespitosus, Chaetomium globosum, Cladophialophora bantiana, Cladophialophora carrionii, Cladosporium cladosporioides, Cladosporium sphaerosperumum, Coniothyrium fuckelii, Curvularia inaequalis, Curvularia lunata, Curvularia senegalensis, Curvalaria verruculosa, Dactylaria constricta var. gallopava, Dissitimurus exedrus, Drechslera biseptata, Exophiala jeanselmei, Exophiala moniliae, Exophiala spinifera, Exerohilum rostratum, Fonsecaea compacta, Fonsecaea pedrosoi, Hormonema dematioides, Lecythophora hoffmannii, Lecythophora mutabilis, Madurella grisea, Madurella mycetomatis, Phaeoacremonium parasiticum (Phialophora parrasitica), Phaeoannellomyces elegans, Hortaea (Phaeoannellomyces) werneckii, Phaeoscleria dematioides, Phialemonium curvatum, Phialemoniium obovatum, Phialophora americana, Phialophora fastigiata, Phialophora repens, Phialophora ricardsiae, Phialophora verrucosa, Rhinocladiella atrovirens, Scolecobasidium constrictum, Scolecobasidium humicola, Scytalidium dimidiatum, Wangiella dermatitidis). Isolates and cultures of Aspergillus spp. can be obtained from the Fungal Genetics Stock Center (University of Kansas Medical Center, Kans.), American Type Culture Collection (ATCC, Manassas, Va.), U.S.D.A. Agricultural Research Service-Fungal databases and specimens (http://nt.ars-grin.gov/fungaldatabases/specimens/specimens.cfm).

The performance of an inhalable formulation of the invention was evaluated using an AERONEB® Pro nebulizer and a cascade impactor. Aerodynamic droplet size distributions were determined using an adapted USP Apparatus 1 nonviable eight-stage cascade impactor (Thermo-Anderson, Smyrna, Ga.) with a spacer. Aerodynamic particle size characterization was conducted twice on the 6.25 mg/mL voriconazole dilution of VFEND® IV. The average total emitted dose (TED) of voriconazole was 25.51 mg over a 20 minute nebulization with a fine particle fraction (FPF, percentage of droplets with an aerodynamic diameter less than 4.7 micrometers) of 71.7% and mass media aerodynamic diameter (MMAD) and geometric standard deviation (GSD) of 2.98 micrometers and 2.192 respectively. The results are listed in Table 1.

TABLE 1
Aerodynamic Particle Size Characterization

| | TED | FPF | MMAD | |
|---|---|---|---|---|
| | (mg) | (%) | (micrometers) | GSD |
| First Test | 29.93 | 73.5 | 2.94 | 2.097 |
| Second Test | 21.09 | 69.8 | 3.03 | 2.288 |
| Average Results of | 25.51 | 71.7 | 2.98 | 2.192 |
| Test 1 and 2 | | | | |
| % RSD¹ | 24.5% | 3.7% | 2.0% | 6.2% |

¹% RSD = Percent Relative Standard Deviation. Aerodynamic droplet size distributions were determined using a USP Apparatus 1 nonviable eight-stage cascade impactor (Thermo-Anderson, Symrna, GA).
TED = total emitted dose.
MMAD = mass median aerodynamic diameter.
GSD = geometric standard deviation.
FPF = fine particle fraction (percentage droplets with an aerodynamic diameter less than 4.7 mm).

The AERONEB® Pro nebulizer produced an aerosol with consistent aerodynamic properties as evidenced by a low percent relative standard deviation (% RSD) for the MMAD and FPF. The TED was variable and prompted the development of a standard operating procedure (SOP) of disassembly, cleaning, and drying of the dosing apparatus and nebulizer between each dose for further studies. The estimated TED, based on measured residual volumes for all dosing during the survival study was 25.44 mg with a % RSD of 3.61%. This indicated the SOP reduced variability in the TED and that mice received consistent dosing during pharmacokinetic and survival studies. The high FPF, >70% was predicted to lead to high lung concentrations of voriconazole, meaning that the inhalable formulation and nebulizer together provide a high percentage of pulmonary delivery upon administration of the formulation by inhalation.

The fungal infection can be a subject's primary or other healthcare concern. It may result from infection outside of a hospital or clinic or it may be a nosocomial infection.

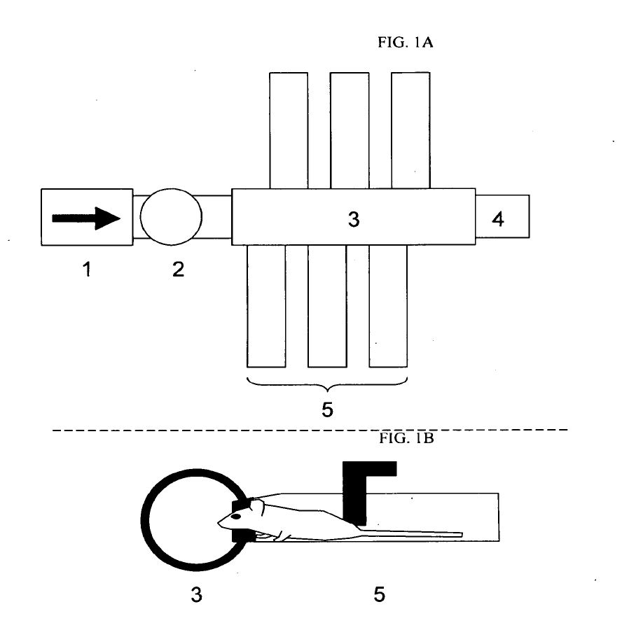

The preliminary safety profile of the inhalable formulation was evaluated in a modified form of an established rodent model. (In Vitro and In Vivo Validation of a High-Concentration Pre-Clinical Rodent Dosing Apparatus for Inhalation, Proceedings of the Annual Meeting of the American Association of Pharmaceutical Scientists, San Antonio, Tex., October, 2006). Rodents were exposed to aerosolized (nebulized) inhalable formulation by employing an apparatus depicted in FIGS. 1A and 1B. The apparatus is adapted to restrain individual rodent in each restraint tube (5) such that the nose of the rodent is located within the spacer chamber (3). A fan (1) blows air in the direction of the arrow through a nebulizer (2) charged with liquid formulation such that aerosolized formulation is passed through the spacer chamber (3) across the nose of each rodent and out to exhaust (4).

A compartment model was used to estimate the PK behavior of aerosolized voriconazole administered to the lungs with absorption from the lungs to a central blood compartment. It was assumed that all the respirable voriconazole was delivered directly to a homogenous lung compartment that could then distribute to the central compartment (blood). Initially, a single dose PK profile was performed on large mice with a 5 L/min flow rate through the dosing apparatus (see Table 2).

TABLE 2
Comparison Between Mice used in
Inhaled Voriconazole Pharmacokinetic Analysis

| | | | | Vfend | |
|---|---|---|---|---|---|
| | | | Mice | Conc. | |
| | | | per | (micro- | Air Flow |
| | Mouse Mass | Lung Mass | Dosing | gram/ | Rate |
| | (g) | (g) | Period | mL) | (L/min) |
| High flow | 31.78 ± 1.21 | 0.233 ± 0.051 | 4 | 6.23 | 5.2-5.4 |
| Rate Mice | | | | | |
| Low flow | 21.75 ± 1.06 | 0.172 ± 0.041 | 6 | 6.09 | 1.0 |
| Rate Mice | | | | | |

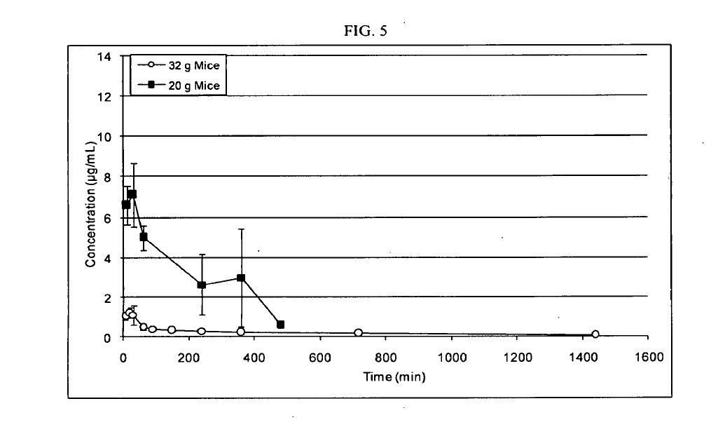

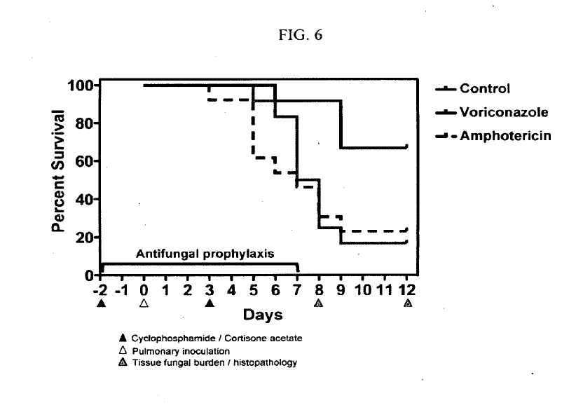

A subsequent PK study was then performed on smaller mice due to the mouse size constraints of the murine model of infection at a lower air flow rate of 1 L/min. Male ICR mice were exposed to a 20 minute nebulization period. Concentrations of voriconazole were determined using 1, 2 or 4 mice per time point. Plasma was separated from whole blood collected to determine drug concentration (adapted from Pascual et al., Antimicrobial Agents and Chemotherapy, 2007, 51:1). Lungs were harvested and homogenized to determine drug concentration (adapted from Lutsar et al., Clinical Infectious Disease, 2003, 37:5). Additionally, the flow rate was reduced to 1 L/min to increase the mouse exposure to atomized voriconazole within the chamber and thereby increase the theoretical dose. FIGS. 4 and 5 include the concentration versus time profiles for voriconazole in the lung and plasma respectively. The relevant PK parameters are detailed in Table 3.

TABLE 3
Inhaled Voriconazole Single Dose Pharmacokinetic Parameters

| | | | | | AUC (0-6 hr) | |
|---|---|---|---|---|---|---|
| | | | Cₘₐₓ | | Lung | Plasma |
| | Tₘₐₓ | | Lung | Plasma | (micro- | (micro- |
| | Lung | Plasma | (micro- | (micro- | gram × | gram × |
| | (min) | (min) | gram/g) | gram/ mL) | min/g) | min/mL) |
| Large Mice | 10 | 20 | 1.62 | 1.18 | 205.3 | 136.4 |
| Small Mice | 30 | 30 | 11.02 | 7.09 | 2408.0 | 1549.8 |

The higher voriconazole concentrations, as observed in the smaller mice, were primarily due to a larger theoretical dose due to a greater voriconazole concentration in the more stagnant cloud of aerosolized API available to be inhaled. The actual difference in mice masses was a minor, but non-trivial contributor to the differences observed in voriconazole concentrations.

The slower flow rate increased the dose delivered to the mice can explain the >10-fold increase in total drug exposure as measured by AUC₀₋₆ for lung tissue and plasma. Similarly, the higher dose led to high lung and blood maximal concentrations (Cₘₐₓ), 11.02 microgram/g and 7.09 microgram/mL, respectively. The Cₘₐₓ of voriconazole, as well as the projected minimum concentrations, were well above the minimum inhibitory concentrations for 90% of isolates (MIC₉₀) for Aspergillus fumigatus of 0.5-1 microgram/mL. The time at which maximal voriconazole concentrations (Tₘₐₓ) were observed in the lung and blood were both within 30 minutes following completion of administration to the lungs. The single-dose pharmacokinetic study determined the Tmax in lung and plasma occurred within 30 minutes after the cessation of nebulization. This was in contrast to the typical Tₘₐₓ in human plasma following a single dose of IV or oral voriconazole was reported as 50-66 minutes following parenteral administration.

The antifungal effects of voriconazole may be maximized through high drug exposure at the site of the infection, the lung tissue, as measured by a rapid Tₘₐₓ with high Cₘₐₓ leading to a high AUC/MIC ratio. Additionally, maintaining prolonged tissue concentrations above the MIC may be beneficial.

The pharmacokinetic profile of inhaled voriconazole demonstrated rapid and extensive distribution of voriconazole from the lung tissue to the central blood. This is a significant improvement compared to the pharmacokinetic properties of another water insoluble antifungal drug reported in the prior art (Vaughn, J M et al. in Eur. J. Pharm. & Biopharm. (2006), 63(2): 95-102 and Vaughn, J M, et al. in Int. J Pharmaceutics. (2007), 338: (1-2), 219-224).

In some embodiments, the ratio of lung Cₘₐₓ to blood Cₘₐₓ following inhalation was determined to be about 1.4-1.6 to 1, indicating extensive distribution of voriconazole from the lung tissue to the blood.

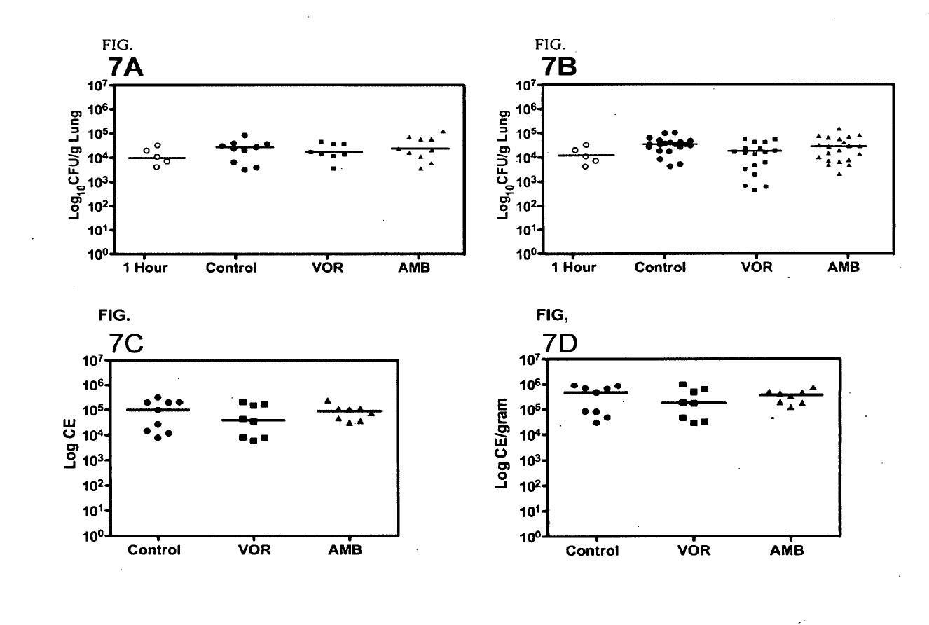

The high and prolonged concentrations of voriconazole in the lung tissue and in the blood suggested clinically significant improvements in survival can occur upon pulmonary administration of a formulation of the invention to a subject having a pulmonary fungal infection. Kaplan-Meier survival curves (Table 4 and FIG. 6) show a statistically significant (p<0.05) difference in survival between the active inhaled voriconazole group and both the negative control group of inhaled sulfobutyl ether-β-cyclodextrin and the positive control group of intraperitoneal Amphotericin B. Survival of A. fumigatus-infected female ICR mice (n=12 for each of three groups) was determined following administration of a voriconazole solution of the invention by 20-minute nebulization using a 1 L/min flow rate. The dose of voriconazole delivered by inhalation BID was about 30-40 mg of voriconazole/kg of body weight. A control sample contained only aqueous carrier and Captisol and it was administered by inhalation BID. Another control sample contained Amphotericin B deoxycholate, which was administered intraperitoneally QD at a dose of about 1 mg/kg of body weight.

TABLE 4
Statistical Analysis of Kaplan-Meier Survival Plot of
A. fumigatus Infected Mice

| | | | Median | |
|---|---|---|---|---|
| | Percent | | Survival | |
| Group | Survival | P-value | (days) | P-value |
| Control | 16.7 | — | 7.5 | — |
| Voriconazole vs | 66.7 | 0.036 | >12 | 4 |
| Control | | | | |
| Voriconazole vs | | 0.047 | | 0.007 |
| AmB | | | | |
| Amphotericin B | 23.1 | 1.0 | 7 | 0.82 |

Survival of A. fumigatus-infected female ICR mice, 20 minute nebulization. 1 L/min flow rate.
N = 12 for each of three groups: VOR = Voriconazole (Inhalation − BID 30-40 mg/kg), Control = Captisol (Inhalation − BID), Amphotericin B deoxycholate (Intraperitoneal − QD 1 mg/kg).

TABLES 5A-5D
Pulmonary Fungal Burden

| | | | |
|---|---|---|---|
| 1 Hour | Control | VOR | AmB |

A - Day 8 Fungal Burden Determined by CFU

| | | | | |
|---|---|---|---|---|
| Median | 3.99 | 4.41 | 4.21 | 4.33 |
| Range | (3.55-4.45) | (3.56-4.91) | (3.62-4.68) | (3.59-5.07) |
| Mean | 4.00 | 4.28 | 4.26 | 4.37 |
| SD | (0.35) | (0.44) | (0.34) | (0.48) |

B - Fungal Burden Determined by CFU for All Mice

| | | | | |
|---|---|---|---|---|
| Median | 3.99 | 4.43 | 4.14 | 4.33 |
| Range | (3.55-4.45) | (3.56-4.91) | (2.60-4.68) | (3.24-5.07) |
| Mean | 4.00 | 4.39 | 3.88 | 4.22 |
| SD | (0.35) | (0.36) | (0.67) | (0.49) |

| | | |
|---|---|---|
| Control | VOR | AmB |

C - Conidial Equivalents Determined by qPCR

| | | | |
|---|---|---|---|
| Median | 4.99 | 4.59 | 4.95 |
| Range | (3.89-5.50) | (3.77-5.32) | (4.49-5.38) |
| Mean | 4.77 | 4.56 | 4.89 |
| SD | (0.21) | (0.23) | (0.11) |

D - Conidial Equivalents per Gram of Lung Tissue
Determined by Qpcr

| | | | |
|---|---|---|---|
| Median | 5.66 | 5.24 | 5.57 |
| Range | (4.47-5.95) | (4.45-5.98) | (5.09-5.88) |
| Mean | 5.35 | 5.19 | 5.50 |
| SD | (0.20) | (0.21) | (0.09) |

All numeric values for CFU and conidial equivalents are given in log₁₀-scale.
VOR denotes voriconazole via inhalation;
Control denotes Captisol via inhalation;
AmB denotes Amphotericin B deoxycholate via intraperitoneal injection.
A - Day + 8 fungal burden as determined by colony forming units (CFU) compared to the fungal burden at 1 hour post infection.
B - Summary fungal burden by CFU for all samples taken, day 8 and day 12 compared with 1 hour post infection.
C - Day + 8 fungal burden by real-time quantitative PCR as measured in conidial equivalents.
D - Day + 8 fungal burden by real-time quantitative PCR as measured in conidial equivalents normalized for wet lung weight.

Approximately 67% of the inhaled voriconazole group survived to the end of the study with a median survival over 12 days. The positive and negative control groups had pronounced decreases in survival with a median survival of 7 and 7.5 days respectively.

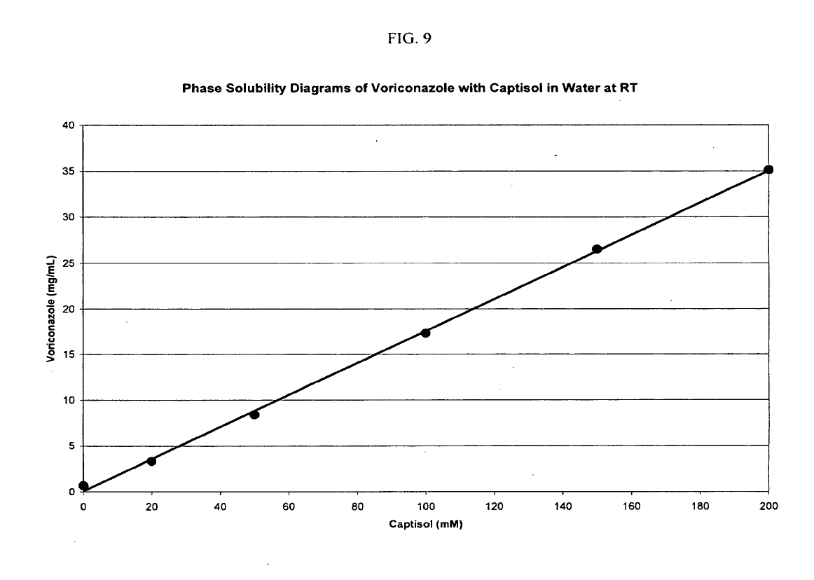

There are no statistical differences between groups although the results trend toward a difference. This discrepancy between survival and fungal burden may be explained by the prevention of disease progression and tissue destruction within the lungs of animals administered aerosolized voriconazole, as demonstrated by the histopathology results. Marked differences in lung histopathology were noted and confirmed between animals administered aerosolized voriconazole prepared according to this invention and those administered intraperitoneal amphotericin B. Invasive hyphae and tissue destruction characteristic of invasive pulmonary aspergillosis were not observed in the lungs of animals that received aerosolized voriconazole (FIG. 8A). In contrast, marked pulmonary disease, including necrosis and the presence of invasive hyphae, was observed within the lung tissue of mice administered amphotericin B (FIG. 8B). These observations are consistent with the inhibition of conidial germination into hyphae by voriconazole. Although CFU and qPCR are markers of tissue fungal burden, they may not be reliable measures of invasive disease as they are unable to distinguish between colonization of the airways by ungerminated fungal spores (conidia) and tissue invasion and disease due to hyphae, which are responsible for damage caused by infection with Aspergillus fumigatus. Thus, the survival advantages (the primary efficacy endpoint) observed with aerosolized voriconazole are supported by the histopathology results (secondary efficacy endpoint).

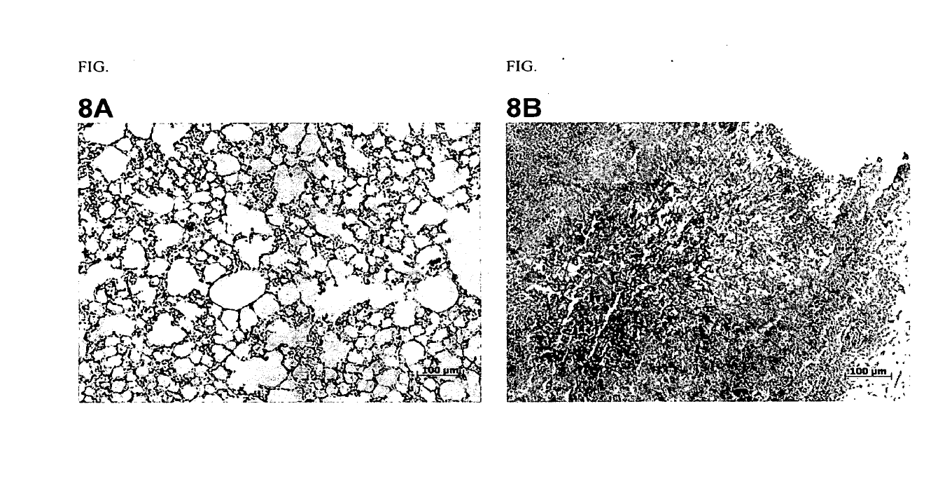

Despite the improved survival and excellent histopathology results of the inhaled voriconazole group, there was no significant reduction in fungal burden between the groups as determined by the number of CFUs per gram of lung mass and CEs as determined by qPCR. (Table 6; FIG. 7A—Day +8 fungal burden as determined by colony forming units (CFU) compared to the fungal burden at 1 hour post infection. FIG. 7B—Summary of fungal burden by CFU for all samples taken, day 8 and day 12 compared with 1 hour post infection. FIG. 7C—Day +12 fungal burden by real-time quantitative PCR as measured in conidial equivalents. FIG. 7D—Day +12 fungal burden by real-time quantitative PCR as measured in conidial equivalents normalized for wet lung mass.)

Increasing the period of time the study was conducted might result in increased fungal clearance for any of the drug treatment groups. This study employed Aspergillus clinical isolate (AF 293). The aerodynamic particle size characterization of diluted VFEND® IV, to 6.25 mg/mL voriconazole, demonstrated a highly consistent aerosol with optimal properties for delivery to the deep lung. The pharmacokinetic profile of a single dose of inhaled voriconazole demonstrated high peak concentrations in the lung and blood as well as rapid and thorough distribution from the lung tissue into the blood. The ratio of lung Cₘₐₓ to blood Cₘₐₓ allowed for comparison of the degree of lung to blood distribution in the published literature. The survival of Aspergillus fumigatus infected mice that received treatment with inhaled voriconazole was significant compared to positive and negative controls. The combined effects of a rapid Tmax, very high Cmax, and rapid distribution from the lung tissue to the blood led to the improved survival and superiority of inhaled VFEND® IV over inhaled particulate itraconazole. These improved pharmacokinetic values are due to the CAPTISOL® (sulfobutyl ether-β-cyclodextrin) and its ability to improve the aqueous solubility of voriconazole and eliminate the dissolution step in particulate itraconazole. The single-dose pharmacokinetic profile suggests insignificant drug accumulation in lung tissue or in plasma.

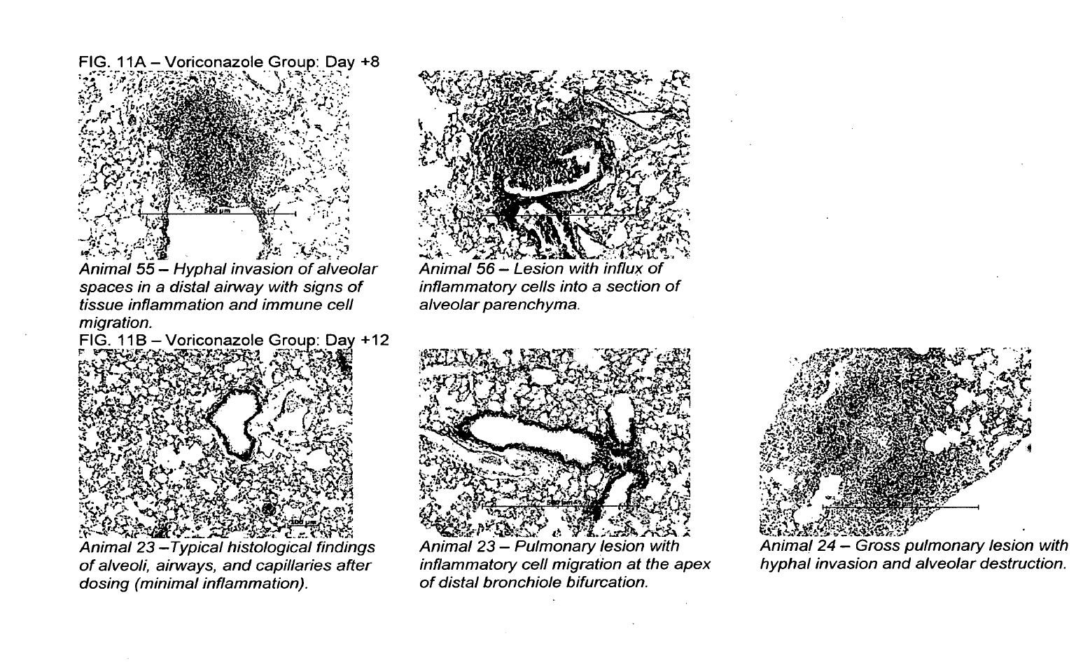

When aerosolized voriconazole was administered to mice following multiple-doses, steady state drug concentrations in lung tissue and blood occurred by the third day of dosing (FIG. 10). Trough voriconazole plasma concentrations were low and had no evidence of accumulation. The trough plasma concentrations remained near or above the MIC₅₀ for A. fumigatus clinical test isolates (0.25 μg/mL) and ranged from 0.177±0.086 μg/mL to 0.325±0.078 μg/mL (mean±standard deviation, below).

### Inhaled Voriconazole Plasma Concentrations Following Multiple-Dose Administration

| | Plasma Concentration ± | Lung Concentration ± |
|---|---|---|
| | Standard Deviation | Standard Deviation |
| | (μg/mL) | (μg/g wet lung weight) |
| Day 3 Trough | 0.218 ± 0.082 | |
| Day 5 Trough | 0.280 ± 0.137 | |
| Day 5 Peak | 2.319 ± 1.515 | 6.726 ± 3.643 |
| Day 10 Trough | 0.176 ± 0.086 | 0.113 ± 0.085 |
| Day 12 Trough | 0.324 ± 0.078 | 0.187 ± 0.225 |

Peak voriconazole concentrations in plasma and lung tissue following multiple doses of inhaled drug were consistent with values suggested from a single-dose of inhaled voriconazole. The peak plasma voriconazole concentration of 2.319±1.476 μg/mL was lower than the concentration associated with toxicity in human studies (6-7 μg/mL) and should therefore correlate with acceptable tolerability. The peak lung voriconazole concentration was 6.726±3.643 μg/g wet lung weight.

The pharmacokinetic profile of inhaled voriconazole suggests blood and tissue drug concentrations promote favorable outcomes. This is due to substantial drug exposure in the lungs at the site of infection as well as in the blood to minimize spreading of the infection.

The ratio of lung Cₘₐₓ to blood Cₘₐₓ following single-dose and multiple-dose administration of inhaled voriconazole was determined to be 1.4-1.6 to 1 and 2.9 to 1. These ratios indicate voriconazole experiences thorough distribution of voriconazole from the lung tissue to the blood following a single-dose but slightly less distribution following multiple-doses (table below).

| Parameter | Value |
|---|---|
| Lung Cₘₐₓ | 11.02 |
| | (single-dose) |
| | 6.73 |
| | (multiple-dose) |
| Blood Cₘₐₓ | 7.09 |
| | (single-dose) |
| | 2.32 |
| | (multiple-dose) |
| Concentration Scale | mcg/mL |
| Ratio of Lung to Blood | 1.6:1 |
| Concentration | (single-dose) |
| | 2.9:1 |
| | (multiple-dose) |
| Lung Tₘₐₓ (min) | 10-30 |
| Blood Tₘₐₓ (min) | 20-30 |
| Source of samples | Animal |
| | (Mouse) |

The high and prolonged concentrations of voriconazole in the lung tissue and in the blood suggested clinically significant improvements in survival may be achieved. The Kaplan-Meier survival curves show a statistically significant (p<0.05) difference in survival between the active inhaled voriconazole group and both the negative control group of inhaled sulfobutyl ether-β-cyclodextrin and the positive control group of intraperitoneal Amphotericin B (Table 4). Approximately 67% of the inhaled voriconazole group survived to the end of the study with a median survival over 12 days. The positive and negative control groups had pronounced decreases in survival with a median survival of 7 and 7.5 days respectively.

Despite the improved survival of the inhaled voriconazole group, there was no significant reduction in fungal burden between the groups as determined by the number of CFUs per gram of lung mass and CEs as determined by qPCR (see Tables 6A-6B, and FIGS. 7A-7D).

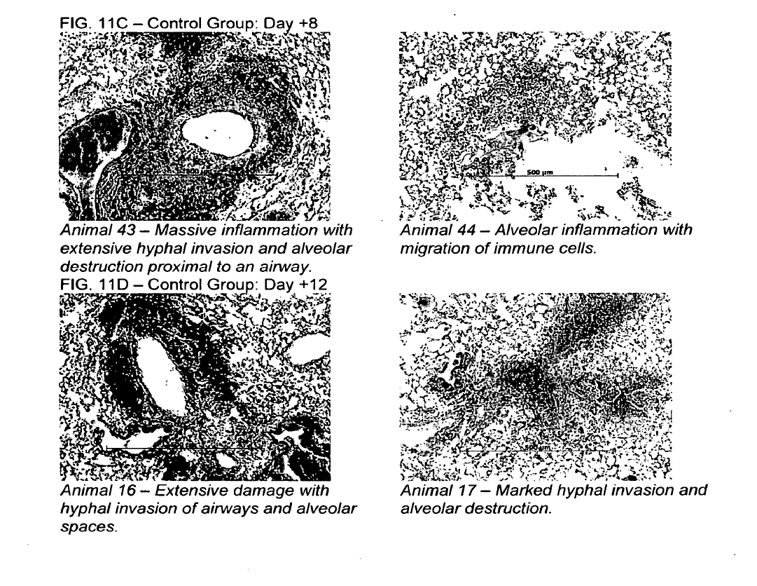

Differences in lung histopathology were observed and quantified for the three treatment groups (Tables 6A and 6B, and FIGS. 11A-11D).

TABLE 6A
Distribution of pulmonary lesions in all available histological samples

| | Control | VRC | AmB |
|---|---|---|---|
| Minimum | 4.0 | 1.0 | 1.0 |
| 25th Percentile | 4.8 | 1.3 | 1.0 |
| Median | 7.0 | 2.0 | 2.0 |
| 75th Percentile | 7.0 | 4.3 | 6.8 |
| Maximum | 7.0 | 5.0 | 8.0 |
| Mean | 6.3 | 2.5 | 3.3 |
| Standard Deviation | 1.5 | 1.7 | 3.3 |

TABLE 6B
Distribution of pulmonary lesions normalized for the number of
lung tissue pieces per slide that were available for evaluation.

| | Control | VRC | AmB |
|---|---|---|---|
| Minimum | 0.4 | 0.1 | 0.1 |
| 25th Percentile | 0.6 | 0.2 | 0.1 |
| Median | 1.3 | 0.4 | 0.3 |
| 75th Percentile | 1.7 | 0.5 | 1.3 |
| Maximum | 1.7 | 0.6 | 1.6 |
| Mean | 1.2 | 0.4 | 0.6 |
| Standard Deviation | 0.6 | 0.2 | 0.7 |

Although lungs from all treatment groups showed evidence of pulmonary lesions, the control and AmB groups were noted to have a larger number of lesions as well as gross abnormalities in lung histopathology. Specifically, lungs from animals that received aerosolized control demonstrated the most severe invasive disease of the small airways, including epithelial disruption, congestion, necrosis, angioinvasion, necrotic foci, and lesions.

The AmB group had similar evidence of lung damage to the control group. However, the distribution of pulmonary lesions in the AmB group was broader than the control and voriconazole groups indicating inconsistent drug action in the lung in the inhibition of pulmonary fungal growth. Therefore, the direct administration of drug to the lungs may eliminate drug action variability due to absorption from the peritoneum and distribution to the lung.

Animals that received inhaled voriconazole had fewer signs of invasive disease and markedly improved histological findings. These animals had evidence of pulmonary lesions but with a narrower and less disperse distribution of lesions than the control or AmB groups. The improved histological findings in the voriconazole group corroborate the hypothesis that inhaled voriconazole suppressed fungal spore germination within the lung to improve survival.

Accordingly, the aerodynamic particle size characterization of diluted VFEND® IV, to 6.25 mg/mL voriconazole, demonstrated a highly consistent nebulized aerosol with optimal properties for delivery to the deep lung. The pharmacokinetic profile of a single-dose of inhaled voriconazole as well as following multiple-doses demonstrated high peak concentrations in the lung and blood as well as rapid and extensive distribution from the lung tissue into the blood. There was an insignificant amount of drug accumulation over 12 days in the lung and blood. The ratio of lung Cₘₐₓ to blood Cₘₐₓ allowed for comparison of the degree of lung to blood distribution in the published literature. The present study differed from the literature in the degree of distribution from the lung tissue into the systemic circulation. The survival of Aspergillus fumigatus infected mice that received treatment with inhaled voriconazole was significantly improved compared to both positive and negative controls. Quantification of pulmonary fungal burden and evaluation of histological lung sections corroborated suppression of conidial germination and growth as the probable mechanism of survival prolongation in this murine model of invasive aspergillosis. The combined effects of a rapid Tmax, very high Cmax, and quick distribution from the lung tissue to the blood led to the improved survival and potential superiority of inhaled VFEND® IV over inhaled particulate itraconazole or other antifungal formulations. These improved pharmacokinetic values are due to the ability of CAPTISOL® (sulfobutyl ether-β-cyclodextrin) to improve aqueous solubility and eliminate the dissolution step following administration of solubilized voriconazole.

In view of the above description and the examples below, one of ordinary skill in the art will be able to practice the invention as claimed without undue experimentation. The foregoing will be better understood with reference to the following examples that detail certain procedures for the preparation of inhalable formulations according to the present invention and to their methods of use. All references made to these examples are for the purposes of illustration. The following examples should not be considered exhaustive, but merely illustrative of only a few of the many embodiments contemplated by the present invention.

### Example 1

The following process was used to make an inhalable aqueous liquid formulation of the invention.

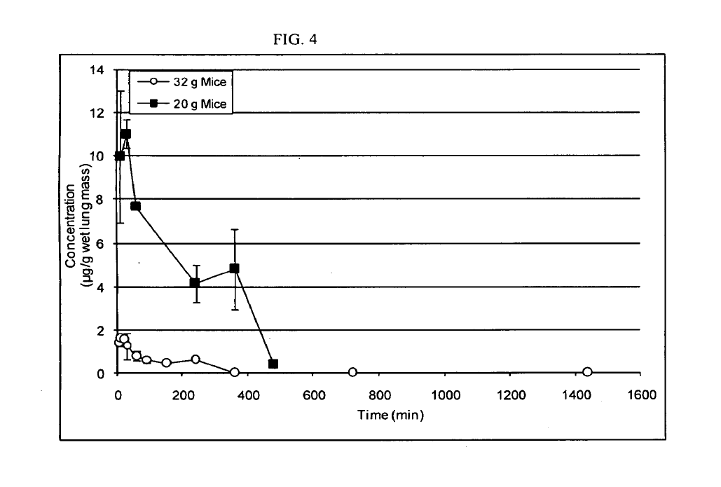

The instructions for use of reconstituted VFEND® IV require further dilution of the product prior to administration.⁹ The osmolality of the reconstituted product (10 mg/mL voriconazole) and dilutions is shown in FIG. 3. The 6.25 mg/mL voriconazole dilution had an osmolality of 293.2 mOsm/kg, the only concentration tested in the isotonic range. The pH of the reconstituted product, 10 mg/mL voriconazole, and the 6.25 mg/mL dilution were determined to be 6.49 and 6.36 respectively. The 6.25 mg/mL dilution was used for further experiments.

### Example 2

The following process was used to determine droplet size for the aerosol generated upon nebulization of a formulation of the invention.

VFEND® IV was reconstituted, diluted, and aerosolized as described previously. Aerodynamic droplet size distributions were determined using a USP Apparatus 1 nonviable eight-stage cascade impactor (Thermo-Anderson, Symrna, Ga.) to quantify total emitted dose (TED) from the nebulizer output, mass median aerodynamic diameter (MMAD), geometric standard deviation (GSD), and percentage droplets with an aerodynamic diameter less than 4.7 micrometer (defined as the percentage fine particle fraction or FPF). The aerodynamic droplet size distribution was conducted as adapted from the guidelines described in USP 30 Section 601: Aerosols, Nasal Sprays, Metered-dose Inhalers, and Dry Powder Inhalers.

### Example 3

The following procedure was used to determine pharmacokinetic parameters following administration of a formulation of the invention with a nebulizer to mice.

Pharmacokinetic studies were performed in male Harlan-Spague-Dawley ICR mice (Hsd:ICR, Harlan Sprague Dawley, Inc., Indianapolis, Ind.). All mice used were handled in accordance with The University of Texas at Austin Institutional Animal Care and Use Committee (IACUC) guidelines and in accordance with the American Association for Accreditation of Laboratory Animal Care.

Mice were dosed using a nose-only dosing apparatus as illustrated in FIGS. 1A-1B. VFEND® IV was reconstituted, diluted, and aerosolized as described previously. The airflow through the dosing apparatus was varied from 1-5 L/min (see Table 2). Sufficient solution was added to the medication reservoir to have residual volume remaining after 20 minutes. The nose-only dosing apparatus and nebulizer were disassembled between each dose, cleaned, dried, and reassembled.

Pharmacokinetic (PK) profiles were determined for high flow rate and low flow rate mice groups following a single 20 minute nebulization. Mice were ordered with masses of 32 g (high flow rate mice) and 20 g (low flow rate mice). Two or more mice were sacrificed by carbon dioxide narcosis at each time point (high flow rate mice—5, 10, 20, 30, 60, 90, 150, 240, 360, 720, and 1440 minutes or low flow rate mice—10, 30, 60, 240, 360, and 480 minutes). Whole blood was collected by cardiac puncture, stored in heparinized vials, and centrifuged to obtain plasma. Surgery was also performed on each mouse to extract the whole lungs which were then homogenized in 1 mL of normal saline. Plasma samples and hung homogenates were analyzed for voriconazole concentration by reverse-phase high-performance liquid chromatography (HPLC). Pharmacokinetic parameters were determined by observation of the voriconazole concentration versus time profiles for plasma and tissue for the time to achieve the maximum concentration (Cₘₐₓ) and the time to achieve the Cmax (Tₘₐₓ). The trapezoidal rule was used to estimate the area under the curve (AUC) for each concentration versus time profile.

### Example 4

The following procedure was used to analyze blood samples for content of voriconazole.

Calibration standards, plasma, and homogenized lung samples were analyzed using similar methods to those previously published. Briefly, sterile water for injection (SWFI) was added to lung tissue and homogenized using a rotor and stator high shear homogenizer. Proteins and other cellular components were precipitated following addition of 0.2M borate buffer (pH 9.0), ethyl acetate, and centrifugation. Supernatant was then extracted three times and liquid was evaporated under a gentle stream of nitrogen. Any residual solids, including voriconazole, were re-dispersed with mobile phase and analyzed spectrophotometrically. Plasma samples had voriconazole extracted through the addition of acetonitrile, centrifugation, and supernatant extraction. Liquid was evaporated under a gentle stream of nitrogen and residual solids, including voriconazole, were re-dispersed with mobile phase and analyzed spectrophotometrically.

Alternatively, voriconazole was extracted from plasma samples through the addition of acetonitrile, centrifugation, and supernatant extraction. The supernatant liquid was evaporated under a gentle stream of nitrogen and residual solids, including voriconazole, were re-dispersed with mobile phase and analyzed spectrophotometrically. For lung tissue, SWFI was added to lung tissue and homogenized using a rotor and stator high shear homogenizer. Voriconazole was extracted from the lung homogenate through the addition of 0.2 M borate buffer (pH 9.0), ethyl acetate, and centrifugation. Supernatant was then extracted three times and liquid was evaporated under a gentle stream of nitrogen. Any residual solids, including voriconazole, were re-dispersed with mobile phase and analyzed spectrophotometrically.

Each sample was analyzed using a Waters Breeze liquid chromatograph (Waters Corporation, Milford Mass.) equipped with a heated (35° C.) JUPITER® C18 (150 mm×4.6 mm, 5 micrometer) with a Universal security guard (Widepore C18) guard column (Phenomenex, Torrance, Calif.). The sample volume was 50 microliter with a UV detection wavelength of 254 nm. The mobile phase consisted of a 50:50 mixture of 0.01 M sodium acetate buffer and methanol at 1 mL/min.

The HPLC assay is sensitive and can provide voriconazole concentrations to the level of specificity indicated herein. Each time point is composed of plasma samples from multiple mice (2, 4, or 6 mice) run in duplicate. There was good intra-sample consistency for each individual animal.

### Example 5

The following procedure was used to culture Aspergillus fumigatus.

Conidia were harvested from Aspergillus fumigatus clinical isolate 293 (AF 293) cultures grown on potato dextrose agar (Hardy Diagnostics, Santa Maria, Calif.) by washing and scraping agar surfaces with 0.1% Tween 80 in sterile physiological saline and filtering through sterile glass wool. Conidia were re-suspended to achieve a final inoculum of ˜1×10⁹ conidia/mL, as confirmed by hemocytometer counts and serial plating.

### Example 6

The following procedure was used to infect the lungs of mice with Aspergillus fumigatus.

Mice were rendered immunosuppressed by intraperitoneal cyclophosphamide (250 mg/kg) and subcutaneous cortisone acetate (250 mg/kg) two days prior to inoculation (Day −2). Both cyclophosphamide (200 mg/kg intraperitoneal) and cortisone acetate (250 mg/kg subcutaneously) were re-administered on Day +3 following inoculation. Mice also received prophylactic antibiotic therapy of ceftazidime 50 mg/kg administered sub-cutaneously on Day −2 through Day +7.

To simulate pulmonary pathogenesis, mice were placed inside an acrylic chamber, and A. fumigatus conidia were introduced by aerosolizing the conidial suspension with a small particle nebulizer (Hudson Micro Mist, Hudson RCI, Temecula, Calif.) driven by compressed air. A standard exposure time of 1 hour was used to allow for complete aerosolization of the conidial suspension. Starting inocula were assessed by colony forming unit (CFU) enumeration from mice one hour post-inoculation.

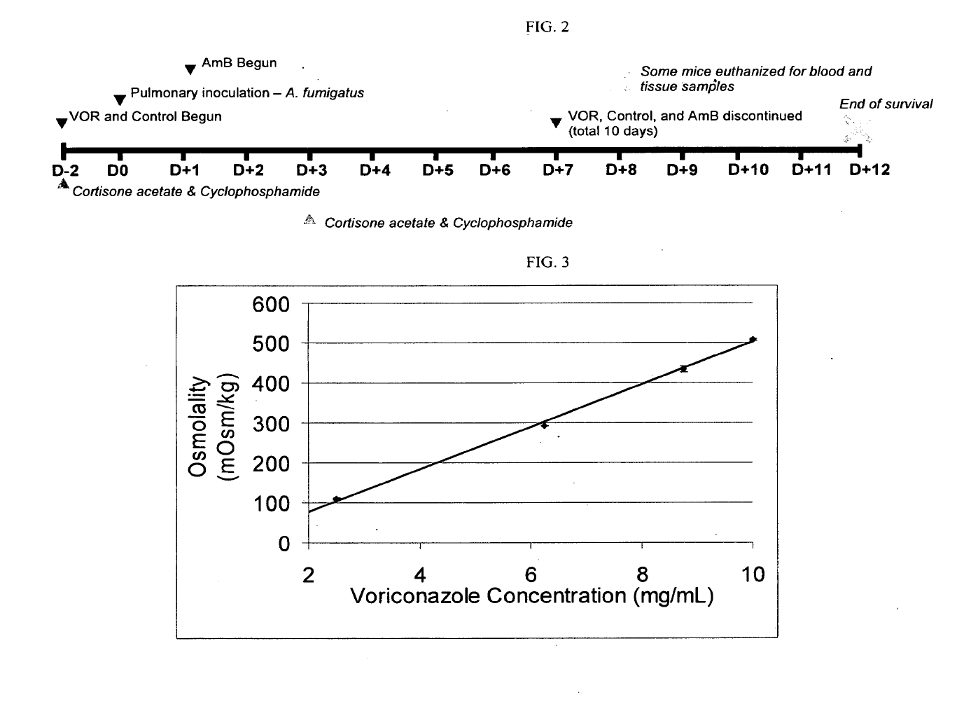

84 mice were randomly assigned equally to three treatment groups: inhaled voriconazole group, inhaled control group, and intraperitoneal AmB. The inhaled voriconazole group received 20 minute nebulizations of diluted VFEND® IV (voriconazole concentration of 6.25 mg/mL) twice daily (BID) beginning on Day −2 and continuing through Day +7. The inhaled control group received 20 minute nebulizations of 100 mg/mL CAPTISOL® solutions BID beginning on Day −2 and continuing through Day +7. The intraperitoneal AmB group received 1 mg/kg Amphotericin B deoxycholate (Apothecon, Princeton, N.J.) by intraperitoneal injection (IP) once daily (QD) on Day +1 and continuing through Day +7 (FIG. 2).

Mice were monitored for an additional 5 days following discontinuation of treatment. Animals that appeared moribund prior to the end of the study were euthanized by halothane and death was recorded as occurring the next day. 12 mice were randomly selected from each group and euthanized on Day +8 for fungal burden analysis while any remaining mice were euthanized on Day +12. 2 additional mice were randomly selected from each group and euthanized on Day +8 and Day +12 for histological analysis

### Example 7

The following procedure was used to determine the pulmonary fungal burden of Aspergillus fumigatus.

Lungs were homogenized in sterile saline (total volume 2 mL) supplemented with gentamicin and chloramphenicol using a tissue homogenizer (Polytron Dispensing and Mixing Technology PT 2100, Kinematica, Cincinnati, Ohio). Serial dilutions were prepared in sterile saline and plated in duplicate onto potato dextrose agar. Following 24 hours of incubation at 37° C., colonies were enumerated and colony forming units (CFU) per gram of lung tissue for each animal were calculated.

Pulmonary fungal burden was also quantified by real-time quantitative polymerase chain reaction (qPCR) using previously described methods. Briefly, DNA was extracted from 90 mL of lung homogenate with the use of a commercially available kit (DNeasy Tissue Kit, Qiagen, Valencia, Calif.) according to the manufacturer's instructions. DNA samples were analyzed in duplicate with the use of the ABI PRISM 7300 sequence-detection system (Applied Biosystems, Foster City, Calif.) with primers and dual-labeled fluorescent hybridization probes specific for the A. fumigatus 1,3-β-glucan synthase (FKS) gene (GenBank accession number U79728). The threshold cycle (Ct) of each sample was interpolated from a six-point standard curve generated by spiking naive mouse lungs with known amounts of conidia (102 to 107). An internal standard was amplified in separate reactions to correct for differences in DNA recovery. The resulting data was expressed as conidial equivalents (CE).

### Example 8

Statistical analyses were conducted as follows. Survival was plotted by Kaplan-Meier analysis, and differences in median survival and percent survival between prophylaxis groups were analyzed by the log-rank test and chi-square test, respectively, using Prism version 4 software (GraphPad, San Diego, Calif.). Differences in fungal burden endpoints (CFU/g and CE) were assessed for significance by analysis of variance with Tukey's post-test for multiple comparisons. A p-value of ≦0.05 was considered statistically significant for all comparisons.

### Example 9

The following procedure can be used to conduct an in vivo trial in animals to demonstrate clinical effect of the method and formulation of the invention for treatment of pulmonary fungal infection of Aspergillus fumigatus.

### Multi-Dose Pharmacokinetic Profile

The pharmacokinetic profile of inhaled voriconazole following multiple doses in mice is investigated to assess dose accumulation through trough voriconazole concentrations in lung tissue and in the blood. This study is conducted in a similar manner to the previously performed pharmacokinetic studies in inhaled voriconazole. Specifically, male ICR mice with an average mass of 20 g are dosed twice daily (BID) with aerosolized VFEND® IV, at a concentration of 6.25 mg/mL, in a nose-only dosing chamber. Trough voriconazole concentrations are assessed at days 3, 5, and 12 while peak voriconazole concentration is assessed on day 5 after dose is initiated.

### Dose Tolerability

A study is conducted to evaluate the tolerability of inhaled voriconazole following a longer period of administered inhaled voriconazole in rats. Tolerability is determined by monitoring of blood chemistry (glucose, liver enzymes, bilirubin, electrolytes, blood urea nitrogen, creatinine, lactic dehydrogenase, and albumin), histological changes in tissues (lung, liver, and kidney), body weight, grooming and appearance, and mortality. Rats are used due to sample requirements for blood chemistry analysis. Male and female Sprague-Dawley rats, with an average mass of 250 g are divided into three groups and dosed BID with inhaled VFEND® IV, at a concentration of 6.25 mg/mL for 10 minutes (low-dose) or 20 minutes (high-dose) or inhaled normal saline for 21 days then followed for 7 additional days post-dosing. Pharmacokinetic peak and trough voriconazole and CAPTISOL® concentrations are assessed in blood and ling tissue at days 7, 14, and 21 after dosing is initiated. Dose tolerability blood tests and tissue samples are assessed at days 7, 14, 21, and 28 after dosing is initiated.

### Physician Sponsored IND

Study 1: A normal volunteer study to assess pharmacokinetics and patient tolerability is conducted. Twelve normal volunteers are given inhaled voriconazole BID for 10 days and pharmacokinetic sampling performed on day 10 (presumed steady state). The pharmacokinetic study may coincide with a methacholine challenge with pulmonary function testing on day 1 and follow up testing on day 10 to determine if airway hyperresponsiveness changed over the 10 day treatment period. A chest x-ray can be obtained at baseline and day 10 to look for changes in lung anatomy (although none should be expected).

Study 2: Performed in patients undergoing a single or double lung transplant. The standard local prophylaxis regiment is inhaled amphotericin B (lipsomal) given immediately after transplantation and for up to 30 days with voriconazole 200 mg BID. Patients are randomized to inhaled amphotericin B or inhaled voriconazole BID for up to 30 days followed by long term voriconazole. Patients are followed for up to 6 months to determine if colonization or infection with aspergillous occurs and bronchoscopy can be obtained as clinically indicated (with an expectation of decline in pulmonary function tests or symptoms of a fungal infection).

### Example 10

### Histological Evaluation of Mice Lungs Obtained During a Prophylaxis/Treatment Study in A. fumigatus Infected Mice

### Materials and Methods

Histopathological changes in lung tissue were evaluated and compared for four mice in each of the inhaled voriconazole, inhaled control, and intraperitoneal AmB groups. The inhaled control was aerosolized CAPTISOL® at a concentration of 100 mg/mL over 20 minutes. On day 8 post inoculation, animals were euthanized using halothane and 10% volume/volume formaldehyde was instilled into the lungs via the trachea. Lungs were then harvested and placed into 10% volume/volume formaldehyde. Tissue was fixed in formaldehyde for an adequate period of time followed by processing and embedding into paraffin wax. Coronal sections of the entire lung were obtained at a thickness of 4-6 μm and mounted on slides. Sections were stained with hematoxylin and eosin and viewed by light microscopy with a Zeiss AxioVision Imager at 10× magnification. Two investigators were blinded and independently evaluated each lung section. The extent of lung damage caused by invasive hyphae was recorded and quantified by counting and normalizing the number of gross lesions.

Normalization of the histopathology results was achieved by using the number of lung pieces (sections) on the slide as a denominator. The total number of lesions on the slide were counted and then divided by the number of pieces of tissue on that slide. This was done because the number of pieces of tissue per slide (again, all from the same cut of lung from the same animal) ranged from 4 to 9. For example, the normalized number of lesions would be 0.4 if 2 lesions were observed on a slide with 5 lung pieces. Similarly, the normalized value would be 0.14 if 1 lesion was observed on a slide with 7 lung pieces.

Differences in lung histopathology were observed and quantified for the three treatment groups. The number of lesions was normalized by the histological slides available for analysis. Although lungs from all treatment groups showed evidence of pulmonary lesions, the control and AmB groups were noted to have a larger number of lesions as well as gross abnormalities in lung histopathology. Specifically, lungs from animals that received aerosolized control demonstrated the most severe invasive disease of the small airways, including epithelial disruption, congestion, necrosis, angioinvasion, necrotic foci, and lesions. The maximum, 75^{th} percentile, median, 25^{th} percentile, and minimum number of normalized lesions in the control group were 1.74, 1.66, 1.29, 0.62, and 0.44 respectively.

The AmB group had similar evidence of lung damage to the control group. However, the distribution of pulmonary lesions in the AmB group was broader than the control and voriconazole groups. The maximum, 75^{th} percentile, median, 25^{th} percentile, and minimum number of normalized lesions in the control group were 1.60, 1.31, 0.28, 0.13, 0.13 respectively. Animals that received inhaled voriconazole had fewer signs of invasive disease and markedly improved histological results. Markedly improved histological results were provided by the formulation and method of the invention as compared to the control sample. “Markedly improved histological results” means that the number and size of the lesions was reduced in the treatment group of animals (VRC) as compared to control animals. These animals had evidence of pulmonary lesions but with a narrower and less disperse distribution of lesions than the control or AmB groups. The maximum, 75^{th} percentile, median, 25^{th} percentile, and minimum number of normalized lesions in the control group were 0.55, 0.51, 0.37, 0.19, and 0.14 respectively.

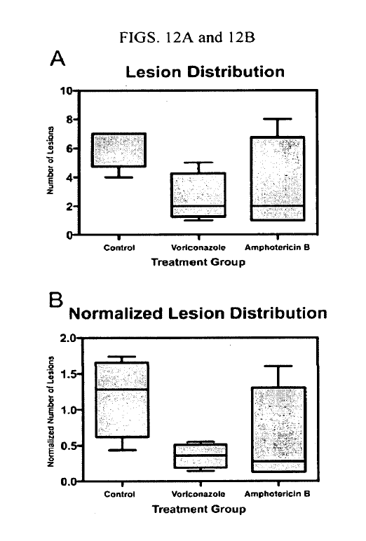

The results are detailed in FIGS. 12A and 12B. The distribution of pulmonary lesions in sections of lung tissue is represented by box plots. The number of lesions identified by the middle two quartiles (25^{th} percentile to the 75^{th} percentile) is represented by the shaded box with the median value as the line within the box. The maximum value is represented by the upper bar while the minimum value is represented by the lower bar. FIG. 12A—The distribution of lesions identified in all available lung sections for each treatment group is represented. FIG. 12B—The distribution of lesions normalized for the number of lung sections available for evaluation for each group is represented.

### Example 11

### Multi-Dose Pharmacokinetic Evaluation in Mice Administered Inhaled Voriconazole

A multi-dose pharmacokinetic profile was performed in male Harlan-Spague-Dawley ICR mice (Hsd:ICR, Harlan Sprague Dawley, Inc., Indianapolis, Ind.). All mice used were handled in accordance with The University of Texas at Austin Institutional Animal Care and Use Committee (IACUC) guidelines and in accordance with the American Association for Accreditation of Laboratory Animal Care. 30 mice, with an average weight of 25.5 grams at the initiation of the study and 28.2 grams at the conclusion of the study, were randomly divided into five groups of 6 mice per group. Each group was dosed twice daily at 08:00 and 16:00 using the nose-only dosing apparatus. VFEND® IV was reconstituted, diluted, and 5 mL was aerosolized twice daily over 20 minutes as described previously. The airflow through the dosing apparatus was 1.0 L/min. The nose-only dosing apparatus and nebulizer were disassembled between each dose, cleaned, dried, and reassembled.

Groups of 6 mice were sacrificed by carbon dioxide narcosis on day 3, 5, 10, and 12 after the initiation of dosing. Trough levels were assessed immediately before the next scheduled dose. Peak levels were assessed 30 minutes after the dose was completed. Whole blood was collected by cardiac puncture, stored in heparinized vials, and centrifuged to obtain plasma. Plasma samples were analyzed in duplicate for voriconazole concentration by reverse-phase high-performance liquid chromatography (HPLC).

On days 3, 5, 10, and 12, the voriconazole plasma trough concentrations (mean±standard deviation) were 0.218±0.083 μg/mL, 0.280±0.137 μg/mL, 0.177±0.086 μg/mL, and 0.325±0.078 μg/mL respectively. There was very little dose accumulation of voriconazole in the plasma when administered to the lung. The trough levels were close to the minimum inhibitory concentration for 50% of Aspergillus fumigatus clinical test isolates. The peak voriconazole concentration of 2.319±1.476 μg/mL (mean±standard deviation) was lower than the concentration associated with toxicity in human studies (6-7 μg/mL).

### Example 12

### Single-Dose Pharmacokinetic Evaluation in Mice Administered Inhaled Voriconazole

Male ICR mice were dosed using a nose-only dosing apparatus as illustrated in FIGS. 1A and 1B. The airflow through the dosing apparatus was varied from 1-5 L/min. VFEND® IV was reconstituted, diluted, and sufficient solution was added to the medication reservoir to have residual volume remaining after 20 minutes. The nose-only dosing apparatus and nebulizer were disassembled between each dose, cleaned, dried, and reassembled.

Single-dose pharmacokinetic profiles were determined in two groups of mice: a high flow-rate group (5 L/min air flow, 32 g average mass) and low flow-rate group (1 L/min, 20 g average mass). Two or more mice were sacrificed by carbon dioxide narcosis at each time point (high flow-rate: 5, 10, 20, 30, 60, 90, 150, 240, 360, 720, and 1440 minutes or low flow-rate mice: 10, 30, 60, 240, 360, and 480 minutes). Whole blood was collected by cardiac puncture into heparinized vials and centrifuged to obtain plasma. Surgery was also performed on each mouse to extract the whole lungs which were then homogenized in 1 mL of normal saline. Plasma samples and lung homogenates were analyzed individually for each animal sampled for voriconazole concentration by reverse-phase high-performance liquid chromatography (HPLC). Concentration values were then averaged to determine the concentration versus time profiles. Pharmacokinetic parameters were determined from the voriconazole concentration versus time profiles for plasma and tissue for the time to achieve the maximum concentration (Cmax) and the time to achieve the Cmax (Tmax). The trapezoidal rule was used to estimate the area under the curve (AUC) for each concentration versus time profile.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

This application claims incorporates the contents of U.S. Provisional Application Ser. No. 61/050,918, filed May 6, 2008, by reference herein in its entirety.

1) A method of treating, preventing or reducing the occurrence of a disease, disorder or condition having an etiology associated with fungal infection or of a disease, disorder or condition that is therapeutically responsive to voriconazole therapy, the method comprising:
administering to a subject in need thereof via pulmonary administration a therapeutically effective amount of voriconazole in an inhalable aqueous liquid formulation comprising voriconazole, sulfoalkyl ether cyclodextrin and an aqueous liquid carrier.

2) A method of treating a fungal infection in a subject comprising:
administering via inhalation to a subject in need thereof a therapeutically effective amount of voriconazole in an inhalable aqueous liquid formulation comprising sulfoalkyl ether cyclodextrin, voriconazole, and aqueous liquid carrier, wherein
the dose comprises 0.5 to 10 ml, 0.5 to 1 ml, 1 to 3 ml, >3 to 6 ml, >6 to 10 ml, 0.25 to 20 ml, 0.1 to 50 ml, or 0.1 to 100 ml of formulation; and
the formulation comprises voriconazole present at a concentration of 1 to 10 mg/ml, 1 to 2.5 mg/ml, >2.5 to 5 mg/ml, >5 to 7.5 mg/ml, >7.5 to 10 mg/ml, 1 to 15 mg/ml, 0.75 to 20 mg/ml, 0.5 to 25 mg/ml, 0.25 to 30 mg/ml, or 0.1 to 50 mg/ml of formulation.

3) The method of any one of the above claims, wherein the formulation is administered via nebulization, and the formulation is completely or partially nebulized over a period of 1 to up to 120 minutes.

4) The method of any one of the above claims, wherein dose provides in the subject a Cmax plasma concentration for voriconazole in the range of about 2 to 8 μg/mL, 2 to 4 μg/mL, >4 to 6 μg/mL, >6 to 8 μg/mL, 1.5 to 10 μg/mL, 1.25 to 15 μg/mL, or 1 to 20 μg/mL.

5) The method of any one of the above claims, wherein dose provides in the subject an AUC for voriconazole in the range of about 1 to 100 μg·hr/mL, 0.5 to 200 μg·hr/mL, 1 to 50 μg·hr/mL, or >50 to 100 μg·hr/mL.

6) The method of any one of the above claims, wherein the formulation provides a Tmax in the lung in the range of about 1-60 min, and a Tmax in the blood in the range of about 5-120 min after administration.

7) The method of any one of the above claims, wherein the fungal infection is a pulmonary fungal infection.

8) The method of any one of the above claims, wherein the sulfoalkyl ether cyclodextrin is compound, or mixture of compounds, of the Formula 1: 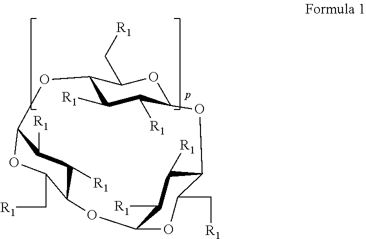
wherein:
p is 4, 5 or 6;
R₁ is independently selected at each occurrence from —OH or -SAET;
-SAE is a —O—(C₂-C₆ alkylene)-SO₃⁻ group, wherein at least one SAE is independently a —O—(C₂-C₆ alkylene)-SO₃⁻ group, preferably a —O—(CH₂)_{g}SO₃⁻ group, wherein g is 2 to 6, preferably 2 to 4, (e.g. —OCH₂CH₂CH₂SO₃⁻ or —OCH₂CH₂CH₂CH₂SO₃⁻); and
T is independently selected at each occurrence from the group consisting of pharmaceutically acceptable cations, which group includes, for example, H⁺, alkali metals (e.g. Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine, ethylenediamine and (C₄-C₈)-cycloalkanolamine among others; provided that at least one R₁ is -SAET.
